(19) United States
(12) Patent Application Publication
(10) Pub. No.: US 2005/0009003 A1
(43) Pub. Date: Jan. 13, 2005

(54) Method and arrangement and computer programme with programme code means for the analysis of neuronal activities in neuronal areas

(76) Inventors: Gustavo Deco, Vilassar de Mar (ES); Norbert Galm, Zorneding (DE)

Correspondence Address: STAAS & HALSEY LLP, SUITE 700, 1201 NEW YORK AVENUE, N.W., WASHINGTON, DC, 20005, US

(21) Appl. No.: 10/492,211

(22) PCT Filed: Aug. 7, 2003
(86) PCT No.: PCT/DE03/02663

(30) Foreign Application Priority Data
Aug. 9, 2002 (DE) 102 36 629.2

(51) Int. Cl. C12Q 1/00; G06F 19/00; G01N 33/48; G01N 33/50

(52) U.S. Cl. 435/4; 702/19

(57) ABSTRACT

Neuronal activities in neuronal areas are analyzed using a coupling model in which coupling model a) the neuronal activities and signals are interconnected by using cross-coupling variables, b) the signals are connected by using signal coupling variables that in each case interconnect two of the signals, c)the neuronal activities are connected by using activity coupling variables that in each case interconnect two of the neuronal activities, in which case at least the signal coupling variables are determined for the analysis when optimizing.

### CROSS REFERENCE TO RELATED APPLICATIONS

This application is based on and hereby claims priority to PCT Application No. PCT/DE03/02663, filed Aug. 7, 2003 and German Application No.10236629.2, filed Aug. 9, 2002, the contents of which are hereby incorporated by reference.

### BACKGROUND OF THE INVENTION

The invention concerns an analysis of neuronal activities in neuronal areas, for example, neuronal activities in the nerve structures in the brain tissue of a patient.

Analyzing neuronal activities and the resulting knowledge about the functionality of a neuronal area as well as via an interaction of neuronal areas form the basis for a functional nuclear tomography or fMRI technology A. W. Toga and J. C. Maziotta (Hrsg), “Brain Mapping: The Methods”, Chapter 9: M. S. Cohen: “Rapid MRI and Functional Applications”, Academic Press 1996 (“the Toga et al. reference”) which is a further development of the known magnetic resonance tomography.

The previously known magnetic resonance tomography (also nuclear magnetic resonance tomography, in short: MR) is an imaging method which generates sectional views of the human body without using stressful X-rays.

Instead, the MR uses the behavior of human tissue in a strong magnetic field. Pathological changes to the human tissue, for example, in the brain or spinal marrow can be detected with this.

Functional disorders in the human tissue, particularly in the brain of a patient can however not be detected with known magnetic resonance tomography.

This is executed by the functional nuclear magnetic resonance tomography or the fMRI technology.

The fMRI technique makes it possible to indirectly measure the neuronal activity in areas of the brain of a patient.

The so-called BOLD signal (blood oxygenation level) is measured in individual areas of the brain which is linked to the neuronal activity in the specific areas.

There are dependencies between the neuronal activities in the areas which, amongst others, result from structures in the brain, i.e. from the neuronal links of nerve cells or nerve structures.

The result of the fMRI measurements shows the course of the activity of the individual areas over a certain period, for example, during cognitive sequences as a result of specific perception processes or motor tasks.

Therefore, in this case, functional disorders in the brain are implicitly contained in the measured fMRI signals.

By using mathematical methods, the fMRI measurements are analyzed and as a result conclusions are immediately drawn about functional disorders in a brain and their causes.

These analysis methods such as the analysis method in A. R. McIntosh et al., Structural Equation Modeling and Its Application to Network Analysis in Functional Brain Imaging, Human Brain Mapping, 2:2-22, 1994, which usually generate statistical characteristic quantities such as statistical correlations between fMRI measurements in different brain areas are mostly based on mathematical models of the brain, particularly the interaction of the brain areas or activities as well as assumptions about static distributions of activities and their influencing parameters.

A further known analysis method is described below.

In the case of this analysis method, a data point s=sₜ gives the average of the totality of all the BOLD signals s1, . . . , sN of the individual n areas at a point in time t or via a time interval t (t=[1;T]).

The fMRI measurement includes many such data points for different perception processes and/or characterizes motor tasks for which the corresponding BOLD signals were possibly measured.

For the known analysis method, not only the individual data points s1, s2, . . . , sT, but statistical characteristic quantities resulting from these are evaluated directly.

For a statistical distribution of the data points s1, s2, . . . , sT it is assumed that they are described by a multivariant normal distribution, i.e. a statistical distribution of the first order with an average μ and a covariance Σ:

$$P ( s \vert μ , Σ ) = \frac{1}{{\sqrt{2 π}}^{N} \cdot \vert Σ \vert} \cdot {ⅇ}^{- \frac{1}{2} {( s - μ )}^{′} {\sum }^{- 1} ( s - μ )} \qquad ( 1 )$$

For sufficiently long readings, the occurrence of the individual data points si of s1, s2, . . . , sT can be considered as statistically independent.

The probability P=P(s1, . . . , sT| μ,Σ) for an occurrence of all measured data points s1, . . . , sT can therefore be described as:

$$\begin{matrix}P ( {s}_{1} , … , {s}_{T} \vert μ , Σ ) = {\prod }_{t = 1}^{T} P ( {s}_{t} \vert μ , Σ ) \\ = \frac{1}{{\sqrt{2 π}}^{NT} {\vert Σ \vert}^{T}} \cdot {ⅇ}^{- \frac{1}{2} {\sum }_{t = 1}^{T} ( {s}_{t} - μ ) ′ {\sum }^{- 1} ( {s}_{t} - μ )}\end{matrix} \qquad ( 2 )$$

Therefore, the unknown variables, the average μ and the covariance Σ only depend on a (brain) model which describes the measured data.

The model assumes a linear statistical connection between the individual BOLD signals:

$${s}_{i} = {\sum }_{j = 1}^{N} {S}_{i j} {s}_{j} + {ɛ}_{i} where i = 1 , … , N or s = S s + ɛ \qquad ( 3 )$$

in which case ε describes the external influence on the individual BOLD signals in the same way as a sensor technique input of sense cells on the tested areas of the brain.

The influence parameters εi and εj on different investigated areas i and j can then be correlated throughout.

The model parameters to be specified are thus the coupling strengths Sᵢ of the basic coupling matrix S, the average με of the external influence ε and the covariance Σε of ε.

The average μ and the covariance Σ depend on these:
μ=μ(S,μ_{ε})
Σ=Σ(S,Σ_{ε}) (4)

In the case of the known analysis method, the model parameters are now determined in such a way that the probability P=P(s1, . . . , sT| μ,Σ) given in (2) becomes the maximum for the occurrence of the measured data.

For this, a method (optimization) of a known maximum likelihood estimation T. W. Anderson, An Introduction to Multivariable Statistical Analysis, Chapter 3, John Wiley & Sons, Inc., New York, London, Sydney, 1994 (“the Anderson reference”) is used.

Using the connections (4) in (2) results in an arithmetic expression that depends on the coupling strengths Sᵢ, the average με and the covariance Σε which is maximized by the optimization.

The optimization then leads to the desired coupling strengths Sᵢ between the BOLD signals.

These, on the other hand, make possible the detection of functional connections between different brain areas in specific perception processes or motor tasks (functional connectivity).

The known analysis method has the disadvantage that it is insufficiently accurate, i.e. it only insufficiently describes the interaction of neuronal areas and therefore possibly leads to incorrect conclusions for the analysis.

One of the causes to which this disadvantage in the case of the known method can be traced back is the fact that a modeling of the functioning of neuronal areas which forms the basis of this method only insufficiently describes a reality, i.e. the biological example or the real brain.

From Beschreibung für eine Software “fmri.pro” zur quantitativen fMRI-Analyse, erhältlich am 07.09.2001, unter http://www.med.uni-muenchen.de/radin/html/arbeitsgruppen/fmri/ccfmri.html (Description of a software “fmri.pro” for the quantitative fMRI analysis, obtainable on 07.09.2001 under http://www.med.uni-muenchen.de/radin/html/study groups . . . ), a software tool for an fMRI analysis method, an “fmri.pro” is known. From Beschreibung fMRI—Gerät, erhältlich am 07.09.2001, unter http://www.unipublic.unizh.ch/campus/uni-news/2001/0147/fmri.html (description fMRI device, obtainable on 07.09.2001, under http://www.unipublic.unizh.ch/campus/uni-news/2001/0147/fmri.html), a device for executing the fMRI technique is known.

### SUMMARY OF THE INVENTION

One possible object of this invention is to specify an improved modeling of the functioning of neuronal areas and with that an improved analysis method by which the neuronal activities can be described or analyzed better than with the known analysis method of neuronal activities.

The inventors propose a method and a system as well as by a computer program with program code and the computer program product for analyzing neuronal activities in neuronal areas.

The method for analyzing neuronal activities in neuronal areas uses for the analysis the signals as well as a coupling model describing the neuronal activities in which case
a) the neuronal activities and signals are interconnected by using cross-coupling variables,
b) the signals are connected by using signal coupling variables that in each case interconnect two of the signals,
c) the neuronal activities are connected by using activity coupling variables that in each case interconnect two of the neuronal activities.

For the analysis, the signals are determined in which case one signal describes the neuronal activity in one of the neuronal areas in each case. Probabilities for an occurrence of the signals are determined in which case the occurrence of signals is based on a statistical distribution described by a normal distribution. Subsequently, the probabilities are optimized by using the coupling model in which case at least the signal coupling variables are determined. The neuronal activities are then analyzed by using at least the signal coupling variables.

The arrangement for analyzing neuronal activities in neuronal areas uses for the analysis the signals describing the neuronal activities as well as a coupling model in which case
a) the neuronal activities and signals are interconnected by using cross-coupling variables,
b) the signals are connected by using signal coupling variables that in each case interconnect two of the signals,
c) the neuronal activities are connected by using activity coupling variables that in each case interconnect two of the neuronal activities.

The arrangement has an analysis unit for the analysis which is designed in such a way that
the signals are determined in which case one signal describes the neuronal activity in one of the neuronal areas in each case,
probabilities for an occurrence of the signals are determined in which case the occurrence of signals is based on a statistical distribution described by a normal distribution,
the probabilities are optimized by using the coupling model in which case at least the signal coupling variables are determined and
the neuronal activities are analyzed at least by using the signal coupling variables.

The inventors recognized that the weak point in the (old) previously known analysis method (relations (1) to (4)) for analyzing neuronal activities is the modeling of linear statistical relations between the signals.

The object of both the old known analysis method and the analysis method is the analysis of neuronal activities by using the signal coupling variables. For this, both methods use signals which represent the neuronal activities in the neuronal areas.

However, in the case of the old known analysis method (relations (1) to (4)), these signals are also equated with the neuronal activities in the linear statistical model (3) used there.

Despite the close collaboration between the signals and the neuronal activities, the equating in the old known analysis method does not apply to the real biological example, but is only a simplified approximation.

The inventors avoid the previous equating method using generally non-linear coupling model, in which the signals are connected to the neuronal activities by using cross-coupling variables.

In addition, the coupling model connects signals by using signal coupling variables that in each case interconnect two of the signals as well as the neuronal activities by using activity coupling variables that in each case interconnect two of the neuronal activities.

As a result, the method can bring about an improved modeling of the functioning of the neuronal areas. The method considerably improves particularly the analysis of neuronal activities and their interaction.

The computer program with program code is equipped to execute all the steps according to the method for analyzing neuronal activities if the program is run on a computer.

The computer program product with program code stored on a machine-readable medium is designed to execute all the steps according to the method for analyzing neuronal activities if the program is run on a computer.

The arrangement as well as the computer program which are designed with program code to execute all the steps according to the method for analyzing neuronal activities if the program is run on a computer, and the computer program product with program code stored on a machine-readable medium which is designed to execute all the steps according to the method for analyzing neuronal activities if the program is run on a computer, are particularly suitable for executing the method for analyzing neuronal activities or one of its further developments explained below.

The further developments described below refer to both the method and the arrangement.

The method described below can be implemented both in the software and the hardware, for example, by using a special electrical circuit.

In addition, the method described below can be implemented by a computer-readable storage medium on which the computer program is stored with program code which implements the method.

The method described below can also be implemented by a computer program product which has a storage medium on which the computer program is stored with program code which implements the method.

Therefore, for a further development in addition to the signal coupling variables both the activity coupling variables and the cross-coupling variables are determined when optimizing.

A method of a maximum likelihood estimation the Anderson reference can be used to execute the optimization in simple way.

When optimizing, an interaction between the coupling model and the probabilities can be taken into consideration as an auxiliary condition.

It is also worthwhile because as a result, the biological example of real neuronal structures can be reproduced in such a way that for the coupling model external influences on the signals and/or neuronal activities are taken into consideration. For example, such external influences can be sensor technique inputs of sense cells on the tested areas of the brain.

The consideration of these external influences can be implemented by using influence coupling variables.

Previous knowledge can also be introduced in the coupling model by the fact that specific coupling variables such as specific signal, cross, activity and/or influence coupling variables are specified according to the previous knowledge.

Therefore, in this way by specifying at least one part of the activity coupling variables, the spatial relations between the neuronal areas can be taken into consideration.

The signals, for example, BOLD signals can be determined by measuring signals or also by transferring and/or reading-in already occurring signals.

The method is particularly suited to the fMRI technique which can considerably be improved as a result of this.

Within the framework of such an fMRI application, the neuronal areas are mostly brain areas with corresponding nerve structures of patients to be tested and diagnosed.

For such an fMRI investigation, the BOLD signals are measured on patients. These BOLD signals describe or represent the neuronal activities in the brain areas. These are evaluated or analyzed in which case the coupling variables are determined.

By using the analysis results, particularly the signal coupling variables, a functional disorder of the patient can be set in the brain area.

### BRIEF DESCRIPTION OF THE DRAWINGS

These and other objects and advantages of the present invention will become more apparent and more readily appreciated from the following description of the preferred embodiments, taken in conjunction with the accompanying drawings of which:

FIG. 1 is a device for executing an fMRI according to an embodiment,

FIG. 2 is a sketch with procedural steps for analyzing BOLD signals according to an embodiment.

### DETAILED DESCRIPTION OF THE PREFERRED EMBODIMENT

Reference will now be made in detail to the preferred embodiments of the present invention, examples of which are illustrated in the accompanying drawings, wherein like reference numerals refer to like elements throughout.

Embodiment: Functional nuclear magnetic resonance tomography (fMRI)

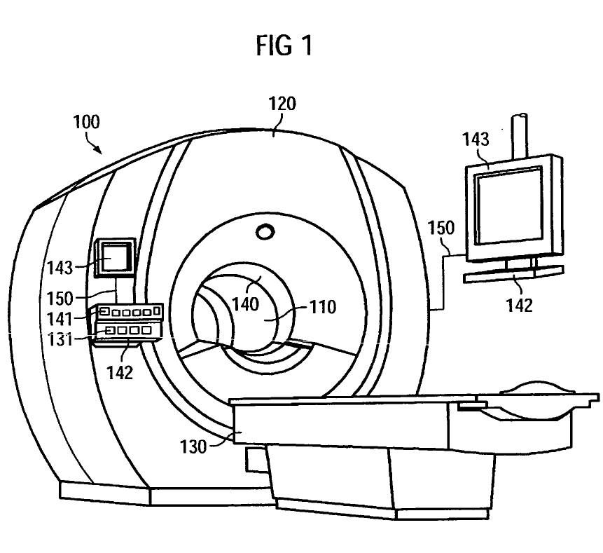

FIG. 1 shows a device 100 for executing a functional nuclear magnetic resonance tomography or magnetic resonance tomography (in short: fMRI), a functional nuclear magnetic resonance tomograph or a magnetic resonance tomograph 100.

The basics of fMRI technology which is a further development of the familiar magnetic resonance tomography are known From the Toga et al. reference.

The nuclear magnetic resonance tomograph 100 shows a closed tunnel 110 which is incorporated in a magnet 120 in such a way that this generates a strong magnetic field in the tunnel 110.

The nuclear magnetic resonance tomograph 100 also shows an examination table 130 that can be introduced into the tunnel 110 on which a patient lies during an examination.

In addition, the nuclear magnetic resonance tomograph 100 has a control unit 131 which allows the examination table 130 to be checked and controlled during the examination, for example, a controlled introduction of the examination table 130 into the tunnel 120.

As a further component, the nuclear magnetic resonance tomograph 100 has a measuring device 140 for measuring BOLD signals (blood oxygenation level dependent), a relevant evaluating device 141 for evaluating the measured BOLD signals, in this case a high-performance computer, as well as an operating or interaction device 142 for the operator and a display device 143 for displaying the result of an examination.

The components of the nuclear magnetic resonance tomograph 100 are interconnected functionally, for example, via signal or data lines 150 via which the data and signals can be sent.

With the functional nuclear magnetic resonance tomograph 100 shown in FIG. 1, the neuronal activity in areas of the brain can be measured, analyzed and a diagnosis can be derived from that on the basis of the fMRI technique.

For that, the measuring device 140 measures the BOLD signal (blood oxygenation level dependent) in individual areas of the brain of the patient which is in collaboration with the neuronal activity in the specific areas.

The result of such fMRI measurements shows the curve of the activity of the individual areas over a certain period in time, for example, during cognitive sequences as the result of certain perception processes or motor tasks which must be carried out by the patient during an examination. Therefore, functional disorders in the brain of the patient are implicitly contained in the measured fMRI signals.

By using the evaluation device 141 which makes available or implements a correspondingly new analysis method, the fMRI measurements, i.e. the BOLD signals measured in individual areas of the brain are analyzed.

As a result, the brain activity is determined as a corresponding activation pattern in the examined areas in the brain and/or the connections between the operating methods of the activation patterns in the examined areas and as a result conclusions are immediately drawn about functional disorders in the brain and their causes.

The new analysis method made available by the evaluation device 140 is based on a model of the brain, the neuron structures in the brain and their behavior, particularly, their interaction on the basis of which the measured BOLD signal is analyzed and evaluated.

The basis of the new analysis method as well as the model of the brain, the neuron structures in the brain and their behavior are explained below.

The results or conclusions of an examination are shown on the display device 143 and can by the operating and interaction device 142 be processed further together with the evaluation device 141. They also serve as the basis for the medicinal diagnosis of an examined patient.

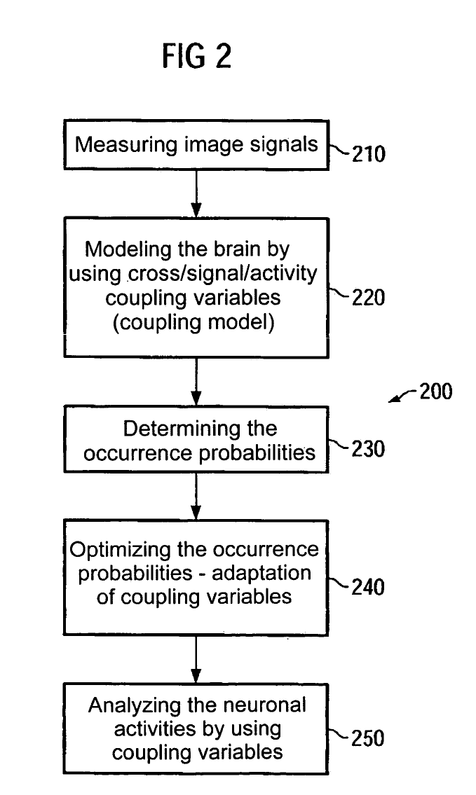

Basis of the new analysis method (FIG. 2, steps 210 to 250)

By using mathematical methods, the fMRI measurements (210), i.e. the BOLD signals in the examined brain areas of a patient are evaluated and analyzed (220-250) and/or compared with reference fMRI measurements and as a result conclusions are immediately drawn about functional disorders in the brain and their causes.

The analysis method 200 that generates statistical characteristic quantities such as statistical correlations between fMRI measurements in different areas of the brain is based on a mathematical model of the brain, particularly, the interaction of the brain areas or activities as well as assumptions on the static distributions of activities and their influence variables (220).

The general principle of this analysis method 200, so-called coupling strengths S which describe the statistical dependencies between the BOLD signals, must be determined in such a way that statistical characteristic quantities which are determined from the fMRI measurements can best be explained (210-250) with this method.

This means that with the desired coupling strengths S, a probability (230) for an occurrence of the measured data, i.e. the fMRI measurement or the BOLD signals should be maximized (240).

Reference is made to the fact that for the new analysis method 200—unless stated otherwise—the relations and assumptions of the old known analysis method (relations (1) to (4)) apply.

A data point s=s(t) represents the a totality of all the BOLD signals s1, . . . , sN of the individual n areas at a point in time t. The fMRI measurement (210) includes a variety of such data points s1, . . . , sT—the BOLD signals of the n areas at different points in time t with 1<t<T (T=maximum number of observed points in time).

The probabilities P=P(s1, . . . , sT| μ,Σ) for the occurrence of all measured data points s1, . . . , sT are determined according to (1) and (2)(230).

Unlike the known analysis method for which the BOLD signals are equated with the neuronal activities in the linear statistical model (3), the new analysis method 200 uses another model, a so-called coupling model (220).

Despite the close collaboration between the BOLD signals and the neuronal activities, equating in the case of the old known analysis method does not apply to the real biological example, but is only a simplified approximation.

The coupling model (220) for the new analysis method 200 considers N BOLD signals (s1, . . . , SN) and M neuronal activities (al, . . . , aM) in which case N=M can be assumed for the same local resolution. In addition, the external influence is implicitly modeled for the coupling model:

$$( \begin{matrix}s \\ a\end{matrix} ) = ( \begin{matrix}S & A \\ B & W\end{matrix} ) ( \begin{matrix}s \\ a\end{matrix} ) + ( \begin{matrix}U \\ V\end{matrix} ) e \qquad ( 5 )$$

Here e designates the statistical independencies of external influences e1, . . . , eP.

Therefore, the parameters of the coupling model (5) are S, A, B, W, U, V, μe and Σe in which case Σe can be assumed diagonally without limitation of the universality.

The coupling model used (5) has a series of advantages. In this way, the measured fMRI data can be explained more precisely. This means, there are model parameters in (5) for which the probabilities from (2) accept higher values than by selecting any of the model parameters in (3) of the old known method and the analysis method described above (relations (1) to (4)).

The explicit modeling of the connections by the coupling model (5) allows a more specific analysis and interpretation of the results:
The signal coupling S between the BOLD signals and the neuronal couplings W between the activities or the areas are distinguished.
Specific assumptions about mutual dependencies between the BOLD signals and the neuronal activities are explicitly taken into consideration in the coupling model. This can be achieved by specific restrictions of A or B.
The external influences e can be characterized better. In this way, specific local influences on the individual areas and global influences can be detected better by the structures in U and V.

The coupling model is written as follows:
sᵢ=S_{α,β}_{ᵢ}⁽ⁱ⁾(s,a,e) für i=1, . . . , N
aᵢ=A_{γ,δ}_{ᵢ}⁽ⁱ⁾(s,a,e) . . . für i=1, . . . , M. (6)

Therefore, the functional connections S⁽ⁱ⁾and A⁽ⁱ⁾can depend on the general parameters α and γ and on the area-specific parameters βᵢ and δᵢ.

The functional connections S⁽ⁱ⁾ and A⁽ⁱ⁾ can be generally accepted, for example, by showing them as a finite series Bronstein-Semendjajew, Taschenbuch der Mathematik, Unendliche Reihen, Funktionenfolgen, Kap. 3.1.14, Seiten 355-375, 22. Auflage, Verlag Harri Deutsch, Thun und Frankfurt/Main, ISBN 3-87 144-492-8, 1985 (Bronstein-Semendjajew, pocket book of mathematics, infinite series, functional sequences, chapter 3.1.14, pages 355-375, 22. Edition, publisher Harri Deutsch, Thun and Frankfurt/Main, ISBN 3-87 144-492-8, 1985 whose coefficients are then also determined as the model parameters by the maximum likelihood estimation (240).

Explicit assumptions can also be made on the functional connections S⁽ⁱ⁾ and A⁽ⁱ⁾. A definite form of A⁽ⁱ⁾ for example results from the formal analysis of the dynamics of neuronal populations based on the models of individual neurons. The resulting model is then as follows:

$$s = A a \\ {a}_{i} = {f}_{{θ}_{i}} ( {\sum }_{j = 1}^{M} {W}_{i j} {a}_{j} + {\sum }_{j = 1}^{P} {V}_{i j} {e}_{j} ) f \ddot{u} r i = 1 , … , M . \qquad ( 7 )$$

Here the BOLD signals s only depend on the neuronal activities a. Spatial relations of neuronal areas can be modeled by restricting A.

On the other hand, in the above case, the activity of an area only depends on the linear summed up total input of this area. Therefore, the remaining parameters θᵢ can for all areas be the same, permanently selected or unknown model parameters or they can differ from area to area in general cases.

In all cases, model (6) generally shows an implicit connection between μor Σ, the unknown parameters for the probabilities (2) and the model parameters to be determined:
μ=μ(α,βᵢγ,δᵢ,μ)
Σ=Σ(α,βᵢ,γ,δᵢ,Σ). (8)

Via this connection, the optimum model parameters can be determined by the maximum likelihood estimation (240).

Contrary to the linear model for the old known analysis method (relations (1) to (4)), the new analysis method carries out the optimization both with the mouel parameters and the parameters μ and Σ of the assumed statistical distribution in which case the equations (8) are taken into consideration as auxiliary conditions.

In the case of the optimization (240), the desired and signal coupling strengths S to be analyzed are then determined between the BOLD signals which describe the connections between the BOLD signals. The signal coupling strengths S are evaluated and analyzed (250) and form the basis of the medicinal diagnosis.

The direct advantage of the new analysis method 200, particularly the coupling model (220) used for this is a more precise analysis of the fMRI data. By setting parameter values with α, βᵢ, γ and δᵢ, the explicit form of the selected relations S⁽ⁱ⁾ and A⁽ⁱ⁾ can also be extracted.

The invention has been described in detail with particular reference to preferred embodiments thereof and examples, but it will be understood that variations and modifications can be effected within the spirit and scope of the invention.

1-18. (cancelled)

19. A method for analyzing neuronal activities in neuronal areas by using signals describing the neuronal activities and a coupling model, comprising:
interconnecting the neuronal activities and signals using cross-coupling variables;
connecting the signals to one another using signal coupling variables, each signal coupling variable connecting two signals;
connecting the neuronal activities to one another using activity coupling variables, each activity coupling variable connecting two neuronal activities;
using each signal to describe the neuronal activity in a corresponding neuronal area;
for each signal, determining the probability for an occurrence of the signal based on a statistical distribution described by a normal distribution;
optimizing the probabilities using a coupling model that determines the signal coupling variables; and
analyzing the neuronal activities using the signal coupling variables.

20. The method according to claim 19, wherein optimizing the probabilities also determines the activity coupling variables and the cross coupling variables.

21. The method according to claim 19, wherein the coupling model considers external influences on the signals and/or neuronal activities using influence coupling variables.

22. The method according to claim 19, wherein previous knowledge is introduced into the coupling model by specifying the cross-coupling variables, the signal coupling variables and the activity coupling variables according to the previous knowledge.

23. The method according to claim 22, wherein spatial relations between the neuronal areas are taken into consideration by specifying at least a portion of the activity coupling variables.

24. The method according to claim 19, wherein the probabilities are optimized using a maximum likelihood estimation method.

25. The method according to claim 19, further comprising optimizing an interaction between the coupling model and the probabilities as an auxiliary condition.

26. The method according to claim 19, wherein the signals are measured.

27. The method according to claim 19, wherein the signals are blood oxygenation level dependent signals.

28. The method according to claim 19, wherein the neuronal areas are areas of the brain of a person.

29. The method according to claim 19, wherein the neuronal activities are analyzed as part of a functional nuclear magnetic resonance tomography analysis of blood oxygenation level dependent signals.

30. The method according to claim 29, further comprising diagnosing a functional disorder in an area of the brain using the blood oxygenation level dependent signals.

31. The method according to claim 20, wherein the coupling model considers external influences on the signals and/or neuronal activities using influence coupling variables.

32. The method according to claim 31, wherein previous knowledge is introduced into the coupling model by specifying the cross-coupling variables, the signal coupling variables and the activity coupling variables according to the previous knowledge.

33. The method according to claim 32, wherein spatial relations between the neuronal areas are taken into consideration by specifying at least a portion of the activity coupling variables.

34. The method according to claim 33, wherein the probabilities are optimized using a maximum likelihood estimation method.

35. The method according to claim 34, further comprising optimizing an interaction between the coupling model and the probabilities as an auxiliary condition.

36. The method according to claim 35, wherein the signals are measured.

37. The method according to claim 36, wherein the signals are blood oxygenation level dependent signals.

38. The method according to claim 37, wherein the neuronal areas are areas of the brain of a person.

39. The method according to claim 38, wherein the neuronal activities are analyzed as part of a functional nuclear magnetic resonance tomography analysis of blood oxygenation level dependent signals.

40. The method according to claim 39, further comprising diagnosing a functional disorder in an area of the brain using the blood oxygenation level dependent signals.

41. A system for analyzing neuronal activities in neuronal areas by using signals and a coupling model describing the neuronal activities, comprising:
a measurement unit to determine signals, each signal describing the neuronal activity in one of the neuronal areas; and
an analysis unit to:
determine probabilities for an occurrence of the signals based on a statistical distribution described by a normal distribution,
optimize the probabilities using a coupling model that determines signal coupling variables that each describe a relationship between two signals, and
analyze the neuronal activities using the signal coupling variables.

42. A computer-readable storage medium storing a program to control a computer to perform a method for analyzing neuronal activities in neuronal areas by using the signals and a coupling model describing the neuronal activities, the method comprising:
interconnecting the neuronal activities and signals using cross-coupling variables;
connecting the signals to one another using signal coupling variables, each signal coupling variable connecting two signals;
connecting the neuronal activities to one another using activity coupling variables, each activity coupling variable connecting two neuronal activities;
using each signal to describe the neuronal activity in a corresponding neuronal area;
for each signal, determining the probability for an occurrence of the signal based on a statistical distribution described by a normal distribution;
optimizing the probabilities using a coupling model that determines the signal coupling variables; and
analyzing the neuronal activities using the signal coupling variables.

43. The computer-readable storage medium according to claim 42 wherein the storage medium comprises a computer-readable data carrier.

44. The computer-readable storage medium according to claim 42 wherein the storage medium comprises a machine-readable carrier.
